(19) United States
(12) Patent Application Publication
(10) Pub. No.: US 2014/0135581 A1
(43) Pub. Date: May 15, 2014

(54) PORTABLE BATTERY POWERED SELF-ILLUMINATED MULTISPECTRAL MULTI-MAGNIFICATION COLPOSCOPE

(71) Applicant: GYNIUS AB, Stockholm (SE)

(72) Inventors: Elisabeth WIKSTROEM SHEMER, Stockholm (SE); Isaac SHEMER, Stockholm (SE); Matthew VOLSKY, Stockholm (SE)

(73) Assignee: GYNIUS AB, Stockholm (SE)

(21) Appl. No.: 13/677,227

(22) Filed: Nov. 14, 2012

(51) Int. Cl. A61B 1/303; A61B 1/06; A61B 1/00

(52) U.S. Cl. CPC A61B 1/303; A61B 1/00195; A61B 1/00032; A61B 1/0638; A61B 1/0676; A61B 1/0684; A61B 1/0646; A61B 1/063
USPC 600/162

(57) ABSTRACT

A portable battery powered self-illuminated multispectral multi-magnification colposcope for visual inspection of the cervix. Includes a housing, an eyepiece, at least one illuminator having two or more wavelength ranges, e.g., white and non-Red, which projects light towards a cervix of a patient. Also includes two or more discrete magnification level paths situated within the housing that pass light reflected from the cervix to the eyepiece when a selected magnification level path is selected by an operator and asserted by a controller. The controller is coupled with the at least one illuminator and the two or more discrete magnification level paths and accepts user gestures via user interface elements to select illumination and magnification settings, wherein the user interface elements may be operated with the hand of the operator that supports the apparatus.

### BACKGROUND OF THE INVENTION

1. Field of the Invention

One or more embodiments of the invention are related to the field of medical instruments known as colposcopes that include illumination and viewing magnification. More particularly, but not by way of limitation, one or more embodiments of the invention enable a portable battery powered self-illuminated multispectral multi-magnification colposcope for visual inspection of the cervix, vagina and vulva.

2. Description of the Related Art

Colposcopes are utilized to detect pre-malignant and other pathological tissue through visual inspection of the cervix, as well as identifying locations for biopsy. Early detection of pre-malignant, malignant and other pathological tissue generally reduces cancer mortality. An appropriate colposcopic examination requires (a) high optical image quality, which allows high resolution at high magnification and (b) high illumination quality in terms of: intensity, homogeneity, color rendering and broad light spectrum. Typical colposcopes are heavy optical devices that are generally not readily portable. The types of colposcopes generally require physical support structures, stands or other mounts. A typical colposcope may weigh 8 kg, or 17 pounds, or more, which is generally too heavy for one-handed operation. In addition, typical colposcopes utilize halogen or other inefficient light sources that require a cooling system and an alternating current electrical source, which further limits their portability. Further, the internal optics are sensitive to mechanical impact and tends to miss align when the colposcope is transported, thus further reducing the transportability of typical colposcopes. These devices allow for detailed visual inspection of the cervix in a relatively fixed location, such as a doctor's office or hospital for example. Hence, patients generally travel to the colposcope based on the portability problems of standard colposcopes. This may occur after an abnormal Pap smear or example or clinical symptoms suggestive of possible malignancy.

Based on their cost, size and stationary nature, colposcopes are mainly utilized in industrialized nations. In third world countries, where access to cervical examinations is limited, cervical cancer represents a high percentage of cancer mortality for women. Hence, portable devices have been developed in an attempt to provide visual examination of the cervix.

Current portable colposcope implementations have limitations with respect to the illumination spectrum emitted, magnification viewing provided, in addition to weight and power limitations. Optical inspection without proper light or magnification is not effective in finding abnormal tissue that may be discovered using heavier, non-portable colposcopes. Hence, known portable colposcopes currently do not provide optimal examinations based on their illumination intensity, illumination homogenicity, color rendering, optical image quality, illumination spectrum, weight, power usage, which limits their efficacy in third world countries.

For at least the limitations described above there is a need for a portable battery powered self-illuminated multispectral multi-magnification colposcope.

### BRIEF SUMMARY OF THE INVENTION

One or more embodiments described in the specification enable a portable battery powered self-illuminated multispectral multi-magnification colposcope that includes a housing that enables a light path generally to travel through the housing. The apparatus is designed to be lightweight and small enough to be portable and operated with one hand of a doctor in general. Embodiments of the apparatus near the eyepiece may be configured to angle downward and away from the doctor's nose and face. The apparatus may output two or more wavelength ranges, generally from the front of the apparatus, wherein light reflected from the cervix of a patient travels back through the device in a “light path”. The light may originate from one or more LED that has an adjustable output frequency spectrum, e.g., that can have different intensities for different output frequency ranges. In or more embodiments this may be implemented with one or more filters placed in front of one or more LED's, for example to allow a white mode and a non-Red mode or to pass Blue and Green ranges, or any other ranges desired. The light path passes through one of two or more magnification paths, for example fixed magnification levels, and the light path exits the apparatus via an eyepiece. Embodiments may be constructed in any size that is generally portable for example pocket-sized with a exemplary length of 166 mm, height of 83 mm, and width of 50 mm, and that weighs 430 g, which is far smaller and more portable than the 8000 g stationary devices known in the art. Embodiments of the invention are not limited to these exemplary dimensions, which are listed to show the dimensions relative to the stationary devices known in the art. The apparatus may include user interface elements to accept inputs to set the frequency spectrum or wavelength range of projected light, the magnification level, and to turn the apparatus off and on. For example, embodiments may also utilize user interface elements such as a mode selector and magnification selector to enable a doctor to operate the illumination and magnification of the apparatus, for example with middle and index fingers of the same hand as that which supports the apparatus.

Embodiments of the apparatus may be implemented with two or more tubes or barrels that receive and pass light that is reflected from a cervix of the patient to the eyepiece when a selected magnification level path is located in the light path. The tubes may be rotated by gears and a motor for example in one or more embodiments, or may be implemented as manually rotatable elements. The barrels may be balanced to weigh approximately the same so that the apparatus is balanced and therefore the center of gravity of the apparatus does not change when the barrels are rotated. This also makes for a more stable apparatus that minimizes overall motion as the barrels turn, which makes for an easier examination by the doctor holding the apparatus in one hand for example. Embodiments may utilize sealed user interface elements for the mode selector and magnification selector to provide a water resistant or water proof enclosure to protect the internal components.

In one or more embodiments, the first of the two or more discrete magnification level paths may utilize a magnification level of 5 power and a second of the two or more discrete magnification level paths may utilize a magnification level other than 5 power. Other embodiments may utilize three or more discrete magnification level paths wherein a first of the three or more discrete magnification level paths includes a magnification level of 5 power and a second of the three or more discrete magnification level paths includes a magnification level of 8 power and a third of the three or more discrete magnification level paths includes a magnification level of 12 power. Any other values may be utilized for magnification levels in two, three or any number of fixed powers greater than three in keeping with the spirit of the invention.

One or more types of power source may be utilized to power the internal components of the apparatus. The internal components that are powered may include a controller and/or buttons. The apparatus may also utilize a status indicator to show status of the device, such as power status, warning or error status, or any other information. Heat sinks may be utilized to cool internal components. Embodiments of the invention allow for charging power source associated with the apparatus via positive and negative charge terminals.

Embodiments may accept an operator gesture respectively and pass the gesture to the controller, wherein the controller is configured to alter the operation of the at least one illuminator between a white mode and a green mode of illumination. For example, in one or more embodiments a magnification of 12 power is selected from the two or more discrete magnification level paths when the green mode of illumination is selected and wherein the magnification is not altered when the white mode is selected via the gesture.

User interface elements may include a first touch sensitive area configured to transmit the gesture to the controller that is configured to increase and decrease light intensity when in a white mode of illumination based on the gesture. One or more embodiments may also include a second touch sensitive area configured to increase and decrease magnification respectively via the controller that is configured to switch between the two or more discrete magnification level paths based on the gesture.

Embodiments may also utilize at least one of the two or more discrete magnification level paths having a curved focal plane configured to approximate a typical curvature of the cervix to provide better viewing of the cervix. The curved focal plane enables better tolerance for movement between the cervix and the device, while still providing a focused image in the eyepiece. In one or more embodiments, the curved focal plane is implemented to have a curvature having a radius of approximately 40 cm.

### BRIEF DESCRIPTION OF THE DRAWINGS

The above and other aspects, features and advantages of the invention will be more apparent from the following more particular description thereof, presented in conjunction with the following drawings wherein:

FIG. 1 illustrates a left top perspective view of an embodiment of the invention.

FIG. 2 illustrates a right top perspective view of an embodiment of the invention.

FIG. 3 illustrates a left side view of an embodiment of the invention.

FIG. 4 illustrates a right side view of an embodiment of the invention.

FIG. 5 illustrates a front view of an embodiment of the invention.

FIG. 6 illustrates a rear view of an embodiment of the invention.

FIG. 7 illustrates a top view of an embodiment of the invention.

FIG. 8 illustrates a bottom view of an embodiment of the invention.

FIG. 9 illustrates a view of the internal parts of an embodiment of the invention.

FIG. 10 illustrates a view of the internal parts of an embodiment of the invention with the heat sinks removed for ease of viewing.

FIG. 11 illustrates a view of the internal parts of an embodiment of the invention without the back housing.

FIG. 12 illustrates a top right exploded view of an embodiment of the invention.

FIG. 13 illustrates a top left exploded view of an embodiment of the invention.

FIG. 14 illustrates a front side perspective view of an embodiment of the telescope barrel.

FIG. 15 illustrates a front acute perspective view of an embodiment of the telescope barrel.

FIG. 16 illustrates a rear acute perspective view of an embodiment of the telescope barrel.

### DETAILED DESCRIPTION OF THE INVENTION

A portable battery powered self-illuminated multispectral multi-magnification colposcope will now be described. In the following exemplary description numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the metes and bounds of the invention.

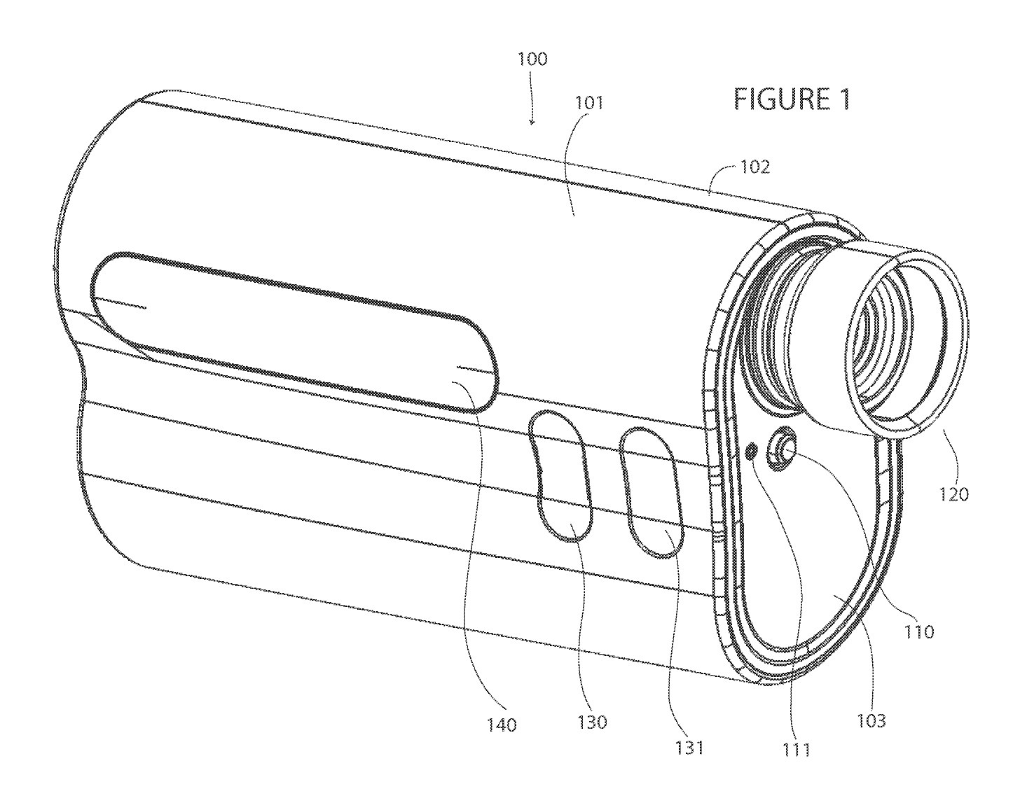

FIG. 1 illustrates a left top perspective view of an embodiment portable battery powered self-illuminated multispectral multi-magnification colposcope 100 that includes a housing, for example as defined by left main housing 101 and right main housing 102. The housing may be constructed from any number of pieces in other embodiments. Embodiments generally include a first end, which is shown on the right side of the figure and a second end, shown on the left side of the figure and a light path between the first end and the second end. The light path generally allows light to travel through the housing. In one or more embodiments, eyepiece 120 is coupled with the first end and configured to enable the light path to be viewed near the first end. As shown, power button 110 accepts inputs to turn the apparatus off and on. Status indicator 111 may be utilized to show status of the device, such as power status, warning or error status, or any other information. For example, if the power is on, the illuminator is on, then status indicator may be set to off, for indicating normal operation. If the status indicator flashes orange, then the battery may be low. If the status indicator flashes orange multiple times, the battery may be too low to operate the apparatus. If the status indicator is on and orange, then the heat sink may be too hot, or there may be an illuminator error. If the status indicator is on and orange for a half second and off for a half second, then the user interface elements may be unplugged or there may be another type of internal fault. If the status indicator is on and green, then the user turned the apparatus on by holding the power button down for 5 seconds to indicate override mode, wherein internal errors or conditions are ignored. If the apparatus is docked for charging, then the illuminator is generally off and the status indicator flashes green to indicate charging or flashes orange to indicate an error. Any other types of gestures or indicator patterns or colors may be utilized in keeping with the spirit of the invention.

Embodiments of the invention may utilize a separate portion, such as back housing 103 to couple with or otherwise mount eyepiece 120, or back housing 103 may be formed as part of left main housing 101 and/or right main housing 102. Heat sink 140 may be utilized to dissipate heat from internal components if desired. Any number of heat sinks may be utilized. Mode selector 130 and magnification selector 131 enable a right-handed operator, such as a doctor to operate the illumination and magnification of the apparatus, for example with middle and index fingers of the same hand as that which supports the apparatus. One or more embodiments may include mirrored buttons on the right side, which is not shown, to accommodate left-handed operation for example.

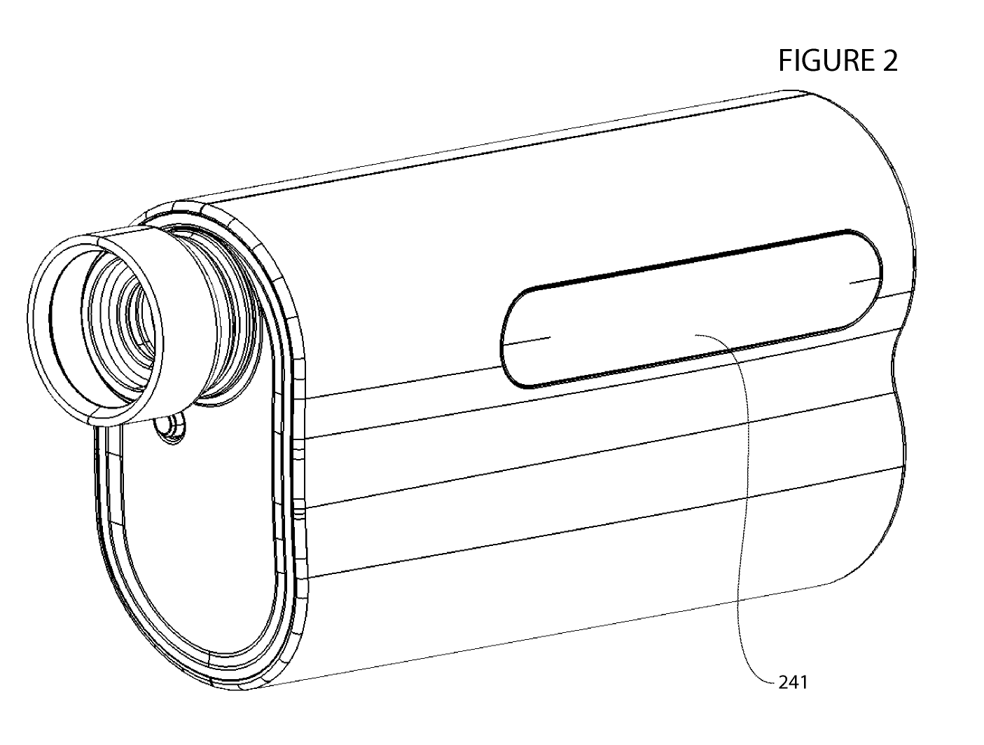

FIG. 2 illustrates a right top perspective view of an embodiment of the invention. As shown, heat sink 241 may be utilized to provide a second heat sink to dissipate heat from internal components within the housing if desired. Heat sinks 140 and 241 are optional in embodiments with low power and low heat internal components, or may be utilized to dissipate heat for other embodiments. Any shape may be utilized for one or more heat sinks if one or more heat sink is utilized. Other embodiments may utilize one or more heat sinks on other portions of the apparatus as desired.

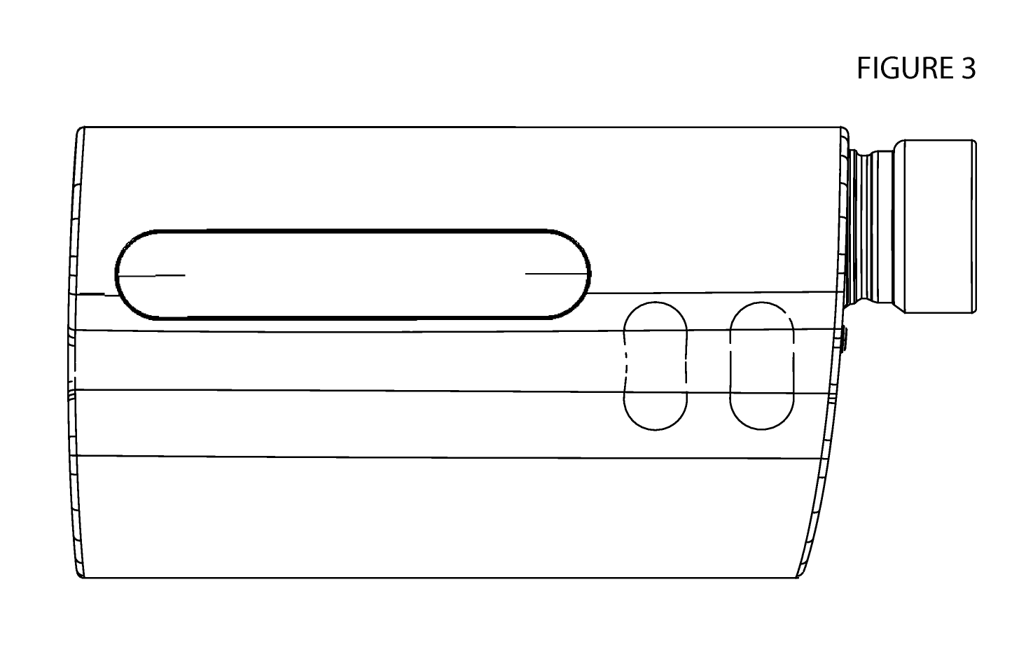

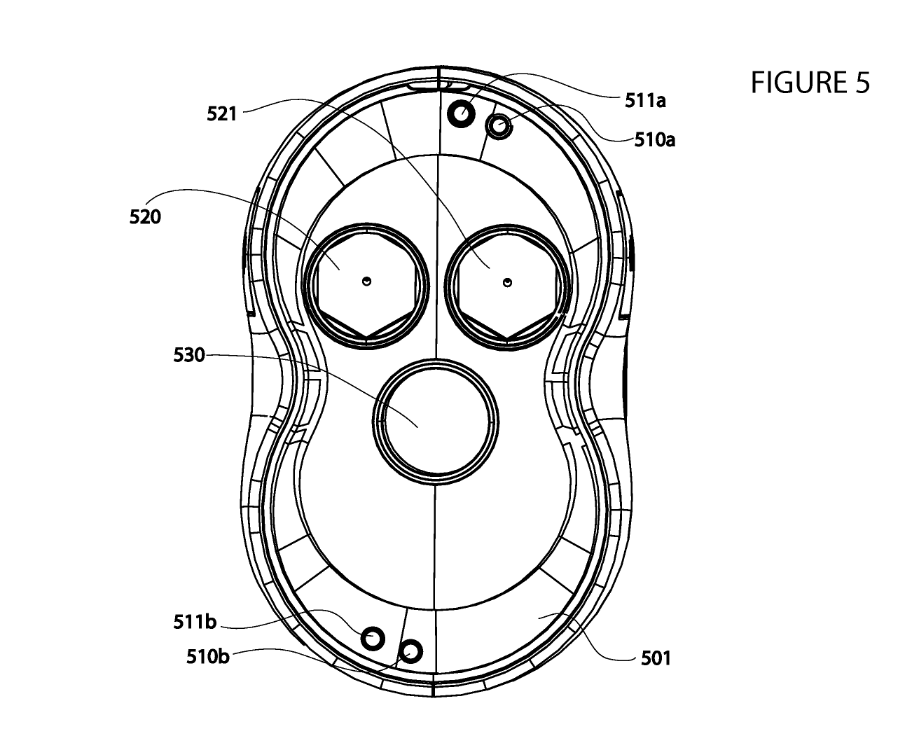

FIG. 3 illustrates a left side view of an embodiment of the invention showing the buttons for right hand operation. The embodiment shown may be constructed in any size, and as shown is constructed optionally with a length of 166 mm and height of 83 mm that weighs 430 g. Embodiments of the invention may be made larger or smaller in one or more dimensions or units of measure as desired, including the width of the apparatus as is shown in FIG. 5, for example 50 mm. Embodiments of the invention are not limited to these exemplary dimensions, but are listed here to show the relative size and weight of one embodiment of the apparatus for completeness. For example, other embodiments may be less than 1000 g, less than 500 g, or any other size, for example less than 200 mm, 100 mm, and 75 mm in length, height and width respectively. Any other dimensions that enable portable use may be implemented in other embodiments of the invention. Although embodiments are constructed to be readily supported and operated manually by a doctor without requiring a support, any mounts or supports may be utilized if desired. As shown the first side, or back housing shown on the right of the figure may be slanted downward and to the left to make room for the doctor's face or nose for example.

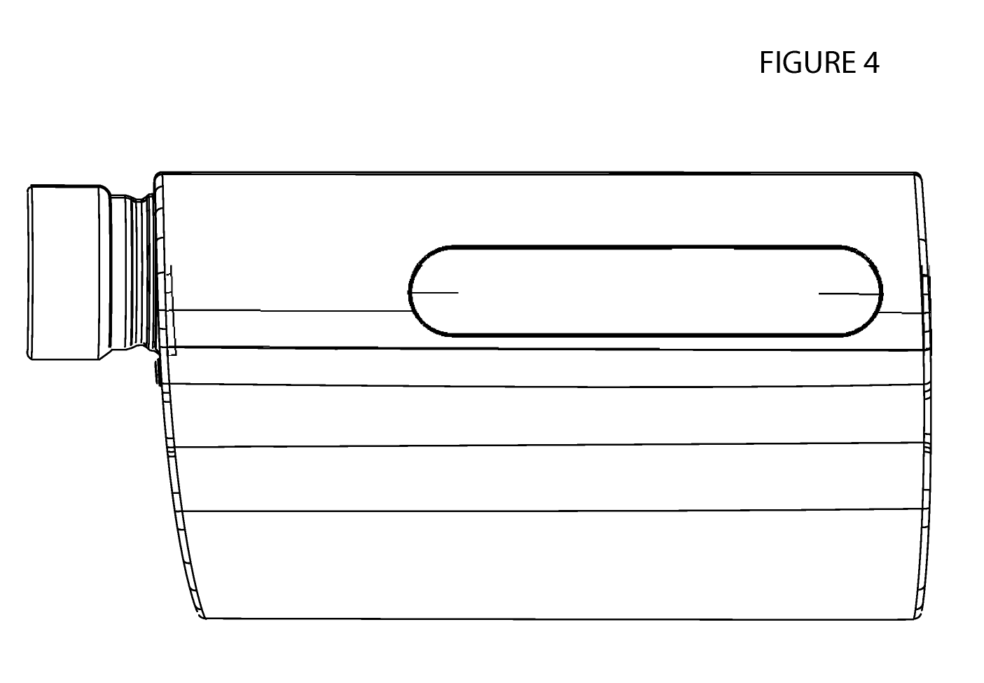

FIG. 4 illustrates a right side view of an embodiment of the invention, which may optionally include buttons if desired for left-handed operation, which are not shown for brevity. If so implemented, the buttons may mirror the buttons or replace the buttons shown in FIG. 3, or alternatively, buttons or other user interface elements may be situated on both sides of the apparatus to facilitate ambidextrous use.

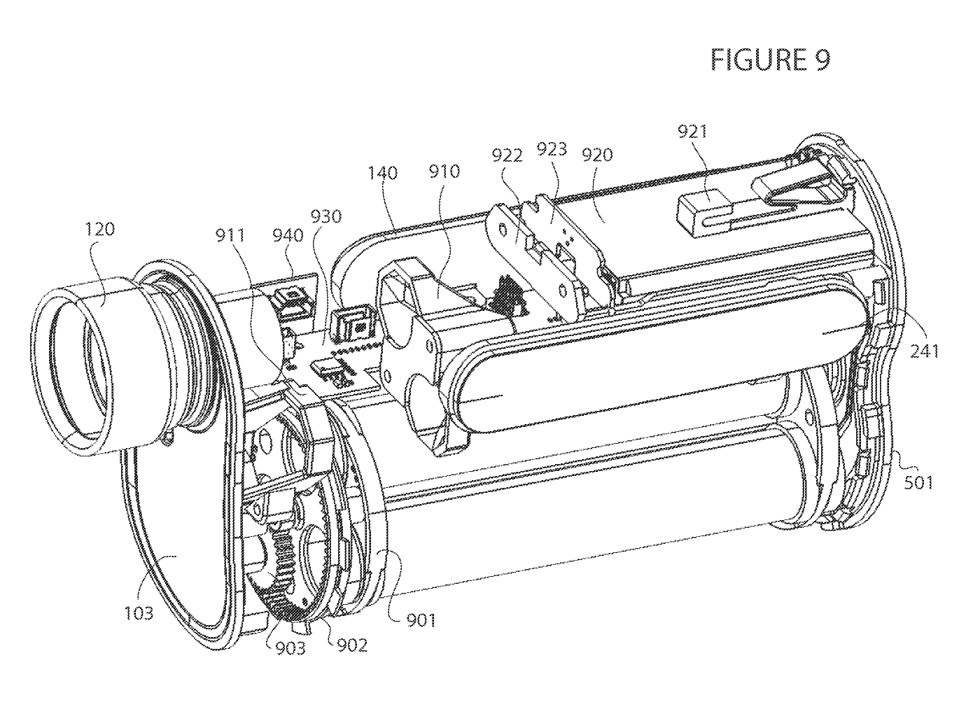

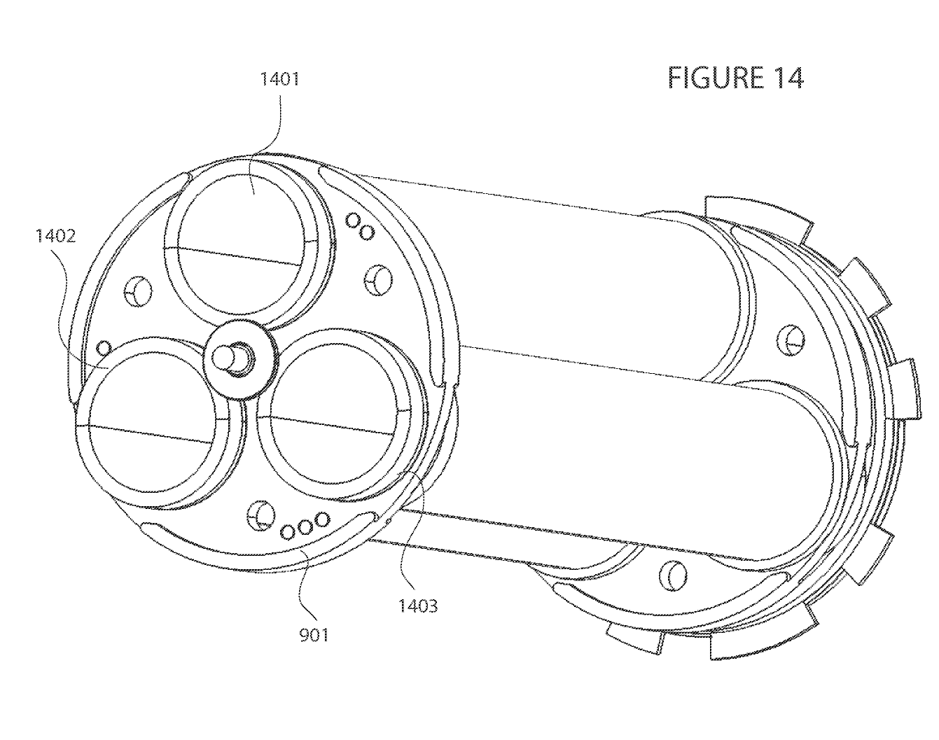

FIG. 5 illustrates a front view of an embodiment of the invention. Embodiments generally include at least one illuminator 520/521 coupled with the second end, (facing out of the figure), wherein the at least one illuminator includes two or more wavelength ranges, or frequency spectrums and wherein the at least one illuminator is configured to project light away from the second end towards a cervix of a patient. Embodiments of the invention may utilize one LED that has a switchable filter to provide two wavelength ranges, or one or two LED's for example with one or more filters coupled with one LED to provide two wavelength ranges, or two LED's with different wavelength ranges without a filter. In one or more embodiments, a filter for Red light, for example about 620 to 740 nm roughly may be implemented in front of one LED, to filter Red light from the output spectrum. Alternatively, or in combination, a filter that passes Blue and Green light may be utilized, i.e., passes roughly 450 to 570 nm. Embodiments of the filter may filter at any attenuation level greater than or equal to 0% or may pass up to 100% of the light produced by the illuminator. Alternatively, or in combination, at least one of the illuminators may be implemented with an adjustable frequency output spectrum to produce two or more ranges of light at different output levels per frequency range. The ranges of Red, Green and Blue are approximate ranges as one skilled in the art will recognize and are not intended to be limiting to an exact value. See also the description for FIG. 14 below for further ranges used in other embodiments. As shown, front housing 501 may be coupled with the left or right main housing or may be an independent piece or coupled with the remaining portion of the apparatus in any other manner. In the embodiment shown, front housing 501 may optionally be indented, for example to a plane or other surface that includes one or more illuminators, such as illuminator 520 for white light and illuminator 521 for green light, or any other spectrum desired and optional glass cover 530 which defines a light path entrance of the apparatus that terminates at the eyepiece on the other end of the apparatus. By indenting front housing 501, protection to the glass cover and illuminators is afforded by providing a vertical offset to these components when the apparatus is stored face down. One or more embodiments may include positive and negative charge terminals 510a, 511a, or alternatively or in combination, positive and negative charge terminals 510b, 511b. The charge terminals may be utilized to charge an internal power supply, such as an internal battery for example when the apparatus is placed in a charging receptacle having corresponding charge electrodes. Embodiments may be implemented to charge at 50 or 60 Hz or both or any other frequency, and at 110V-240V alternating current or any other voltage, whether from a direct current or alternating current source. In one or more embodiments, the power supply may be implemented as a Lithium-ion battery, for example having a battery duration of 2-4 hours, or more or less depending on the intended operational environment. Embodiments of the front housing may be configured to open to allow the battery to be replaced. In other embodiments, any other type of battery or power supply may be utilized. The description of FIG. 9 also illustrates other embodiments of the power supply.

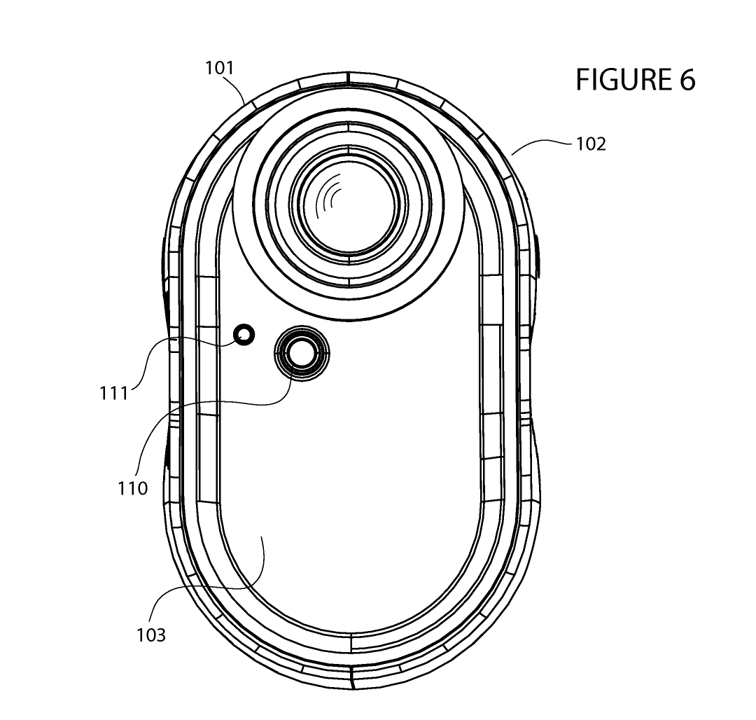

FIG. 6 illustrates a rear view of an embodiment of the invention. As shown, power button 110 is situated on the left side of the apparatus, for example to facilitate operation with the right thumb of a hand while the apparatus is being supported and operated. Embodiments of the invention may utilize modes of operations of mode selector 130 and/or magnification selector 131 alone or in combination to power the apparatus on or off. For example, in one or more embodiments, power button 110 may be eliminated in embodiments that accept one or more inputs, such as mode selector 130 and/or magnification selector 131 being asserted for a given amount of time for example. In other embodiments of the invention utilizing an accelerometer or gyroscope, or proximity sensor, or passive motion switch that requires no power to detect and assert movement, the apparatus may accept motion gestures such as acceleration, or angular movement, or for example shaking left and right to power the device on for example. Any other type of user interface element other than a button may be utilized for example to power the apparatus on or off in keeping with the spirit of the invention and to minimize effort on behalf of the doctor using the apparatus.

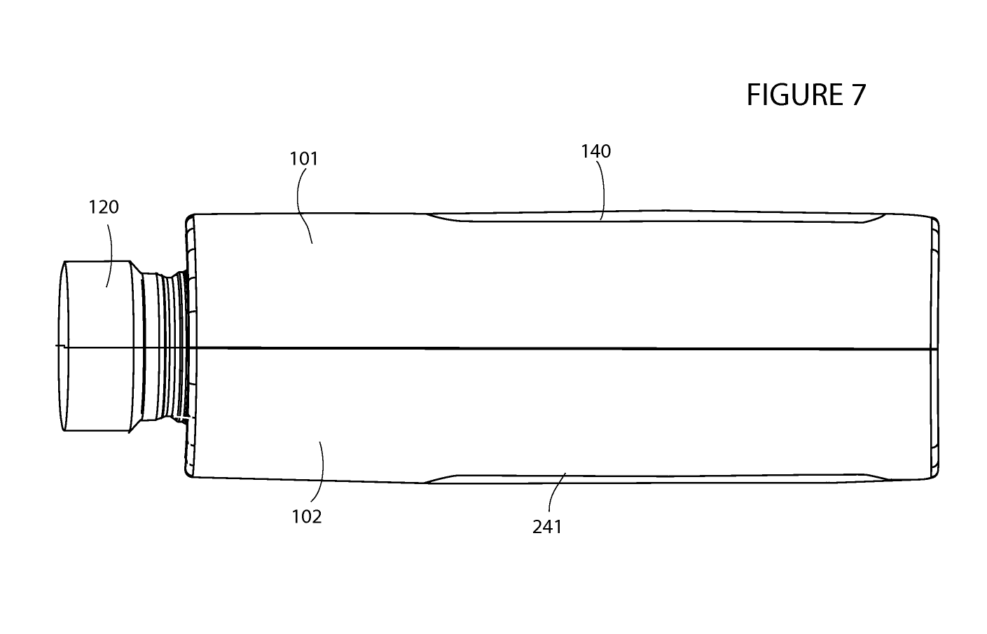

FIG. 7 illustrates a top view of an embodiment of the invention. As shown, two heat sinks are utilized in the embodiment shown, namely heat sinks 140 and 241 although this is not required based on the efficiency of the internal components for example as previously discussed in relation to FIGS. 1 and 2.

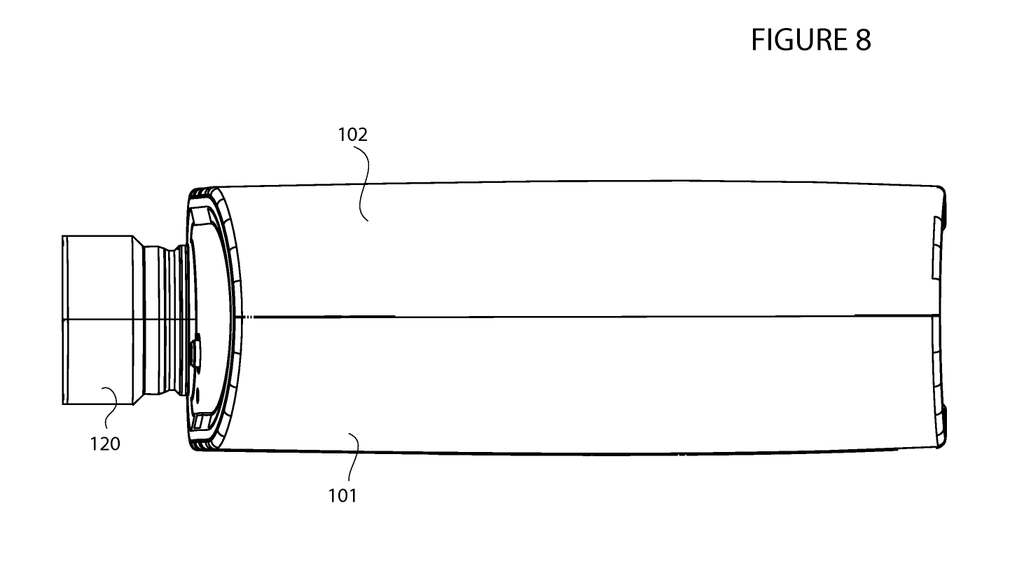

FIG. 8 illustrates a bottom view of an embodiment of the invention. As shown the back housing on the left that supports the eyepiece, may be angled forward to the right as shown, and away for example from the doctor's nose and face as previously discussed in relation to FIG. 3.

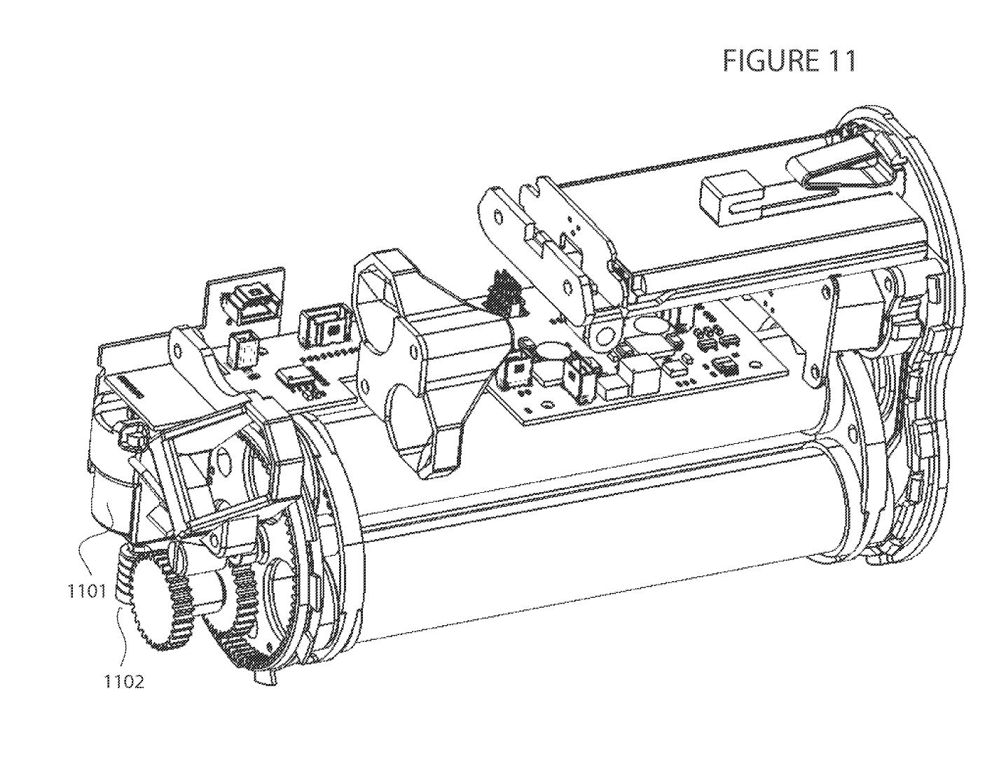

FIG. 9 illustrates a view of the internal parts of an embodiment of the invention. Light enters the apparatus from the second side, or front of the apparatus shown on the right (see FIG. 5 for optional glass cover) and travels through one of the two or more magnification paths, for example one of the tubes (see FIG. 14 for three magnifications barrels or tubes). Each of the two or more discrete magnification level paths situated within the housing are configured to receive and pass light that is reflected from a cervix of the patient to the eyepiece when a selected magnification level path is located in the light path. Although the implementation may utilize tubes holding fixed magnification levels, this is not required and prisms or other optical components including rotatable lenses may be utilized to provide two or more magnifications levels within the light path for example. The tubes shown in this embodiment are held by tube support 901, which is rotationally coupled with the device so as to align one magnification tube at a time in the light path to provide multiple fixed magnifications. Tube support 901 for example may be rotated with ring gear 902, that is rotated via double gear 903, which is coupled with a motor for example (see FIG. 11). Other embodiments may utilize an external lever, for example in place of magnification selector 131, to rotate the magnification path to avoid use of a motor and associated power drain. Embodiments may utilize sealed user interface elements for the mode selector and magnification selector to provide a water resistant or water proof enclosure to protect the internal components. After light passes through one of the magnification tubes, the light reflects off of a prism held by prism clamp 911 which redirects light forward to a prism held by prism clamp 910, which reflects light rearward to eyepiece 120.

Also shown is the power source, namely battery 920, which is held by battery clip 921, in battery housing 922 that holds battery charge pin 923. Any other method or structure may be utilized to hold the power source, internally or externally or both and of any type of power source, for example a fuel cell. Micro energy harvesting may also be utilized to translate mechanical motion of the apparatus into power to recharge the battery for example. Regardless of the type of power source, the power source is utilized to power the internal components of the apparatus. The internal components that are powered may include controller 930 and/or buttons, for example touch switch PCB 940.

One or more embodiments of the invention utilize a microcontroller, or any type of controller coupled with the at least one illuminator and the two or more discrete magnification level paths. The controller is generally configured to receive user input to control the at least one illuminator and the two or more discrete magnification level paths. In one or more embodiments of the invention, after the apparatus is powered on, for example via power button 110, the controller accepts inputs from mode selector 130 and magnification selector 131 and asserts power to at least one illuminator 520/521 to project light forward towards a cervix of the patient. One or more embodiments of the invention power up in 8 power magnification mode with white light wavelength range selected. Light reflecting back through the glass cover 530 enters the magnification path that is in line with the glass cover and otherwise in the light path of the apparatus, which then reflects off of the two prisms held in prism clamps 910 and 911 and passes through eyepiece 120 to reach the eye of the doctor performing the examination. The controller continues to accept inputs and assert illuminators as instructed or alter magnification levels until the power button is asserted again. In one or more embodiments, the apparatus may power off automatically after a timeout when no movement has been detected via an accelerometer for a predefined time period. In other embodiments, if no user inputs are received within a predefined time period via selectors 130 or 131, then the apparatus may power off.

Embodiments of the invention may utilize at least one user interface element configured to accept an operator gesture and pass the gesture to the controller to control the at least one illuminator and the two or more discrete magnification level paths wherein the user interface element may be operated with the hand of the operator that supports the housing. The gesture may alter the operation of the at least one illuminator and the two or more discrete magnification level paths respectively. In other embodiments, the apparatus includes at least two user interface elements configured to accept an operator gesture respectively and pass the gesture to the controller, wherein the controller is configured to alter the operation of the at least one illuminator between a white mode and a green mode of illumination and wherein a magnification of 12 power is selected from the two or more discrete magnification level paths when the green mode of illumination is selected and wherein the magnification is not altered when the white mode is selected via the gesture. One or more embodiments of the at least one user interface element may include a first touch sensitive area configured to transmit the gesture to the controller that is configured to increase and decrease light intensity when in a white mode of illumination based on the gesture. One or more embodiments may also include a second touch sensitive area configured to increase and decrease magnification respectively via the controller that is configured to switch between the two or more discrete magnification level paths based on the gesture.

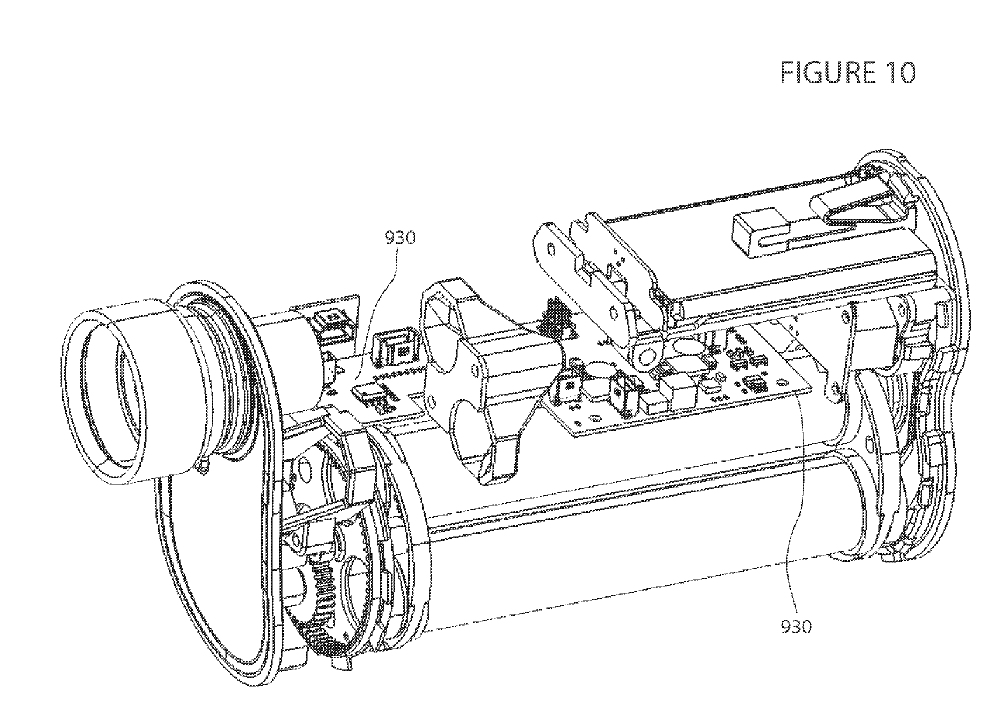

FIG. 10 illustrates a view of the internal parts of an embodiment of the invention with the heat sinks removed for ease of viewing. As shown, controller 930 may be constructed on a PCB and may include a microcontroller chip, memory and any other digital components to obtain inputs from any user interface elements and internal components and provide outputs to any components to control or otherwise interact with any component in the apparatus as one skilled in the art will appreciate.

FIG. 11 illustrates a view of the internal parts of an embodiment of the invention without the back housing. As shown, motor 1101 may be controlled via magnification selector 131 (see FIG. 1), via controller 930 (see FIG. 10), or in any other manner. Motor 1101 as shown couples with worm gear 1102 to turn the double gear 903, ring gear 902 and thus tubes via tube support 901 (see FIG. 9).

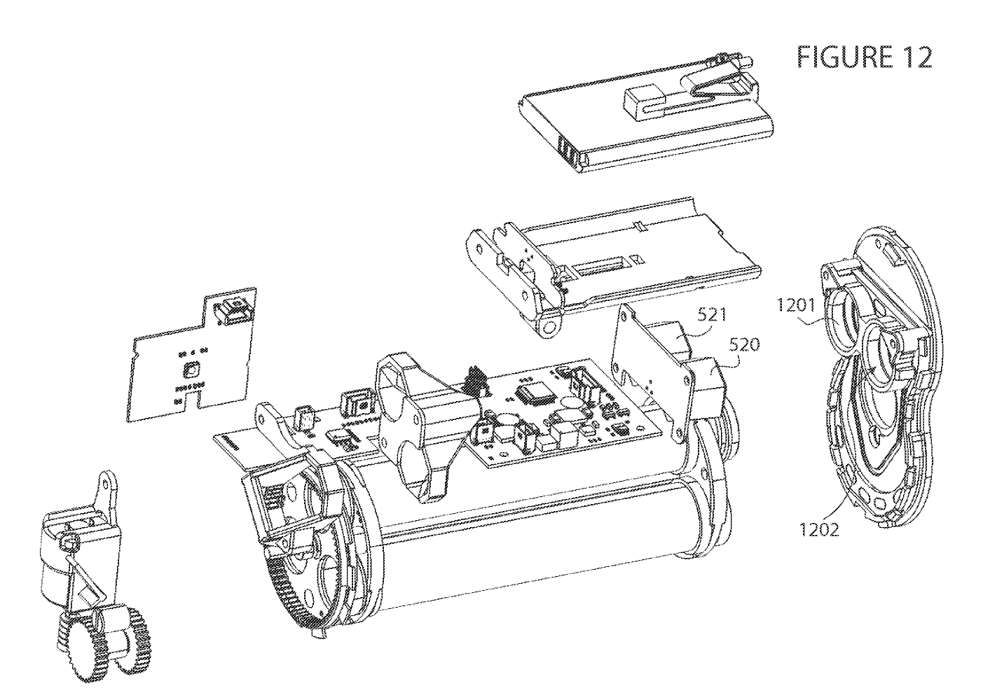

FIG. 12 illustrates a top right exploded view of an embodiment of the invention. As shown, filter 1201 may be utilized to enable a wavelength range or frequency spectrum that differs from that provided by illuminator 521. In one or more embodiments, filter 1201 provides a bandpass green filter. Also shown is glass cover 1202, which may also be implemented with a filter or having a different wavelength range than that of filter 1201, or be implemented with a clear glass cover. Alternatively, embodiments of the invention may utilize one opening with two different frequency spectrum illuminators that are switched or combined to form two frequency spectrums. For example, in one or more embodiments, a filter, either mechanical or electrical may be rotated in front of, or otherwise applied to a single opening with one LED projected through the filter or alternatively through a clear opening or cover, or second filter as desired and in keeping with the spirit of the invention.

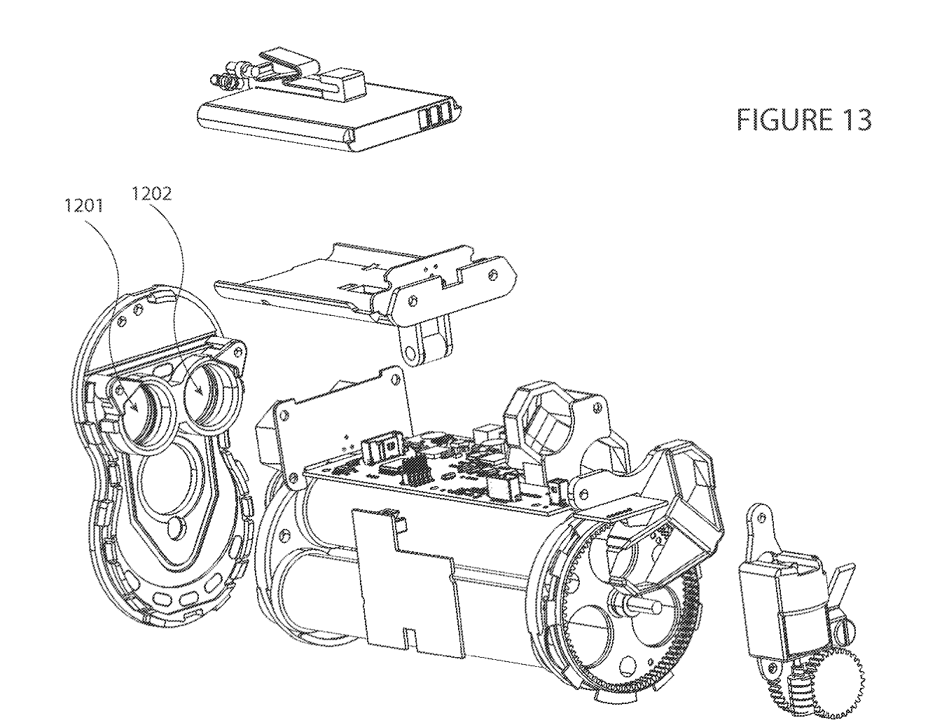

FIG. 13 illustrates a top left exploded view of an embodiment of the invention. Although filter 1201 and cover 1202 are shown coupled with the front housing, they may also be coupled with the illuminators or held in any other manner in front of their respective illuminators.

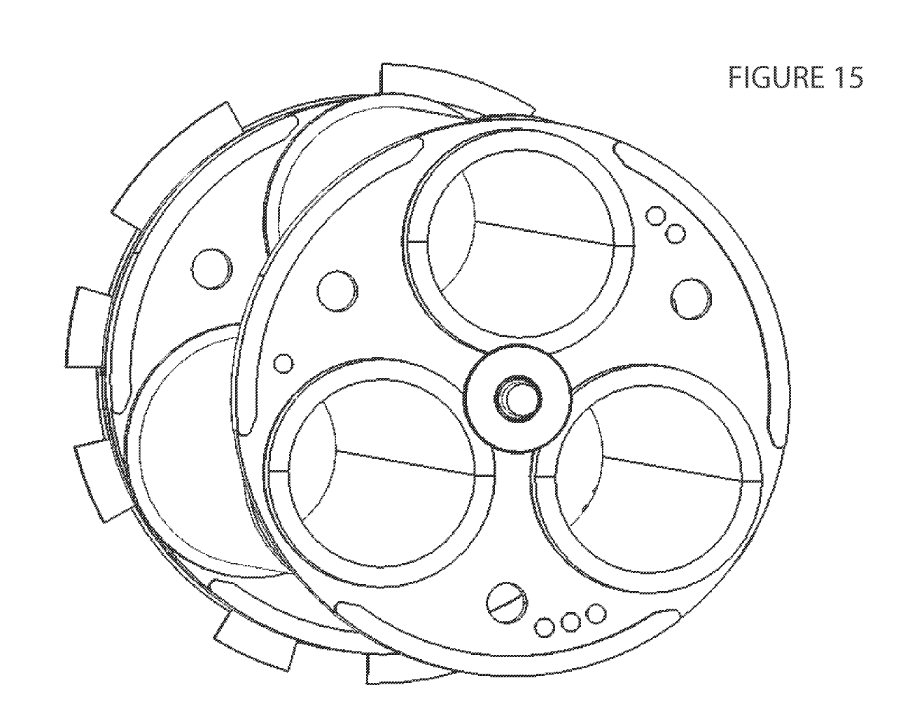

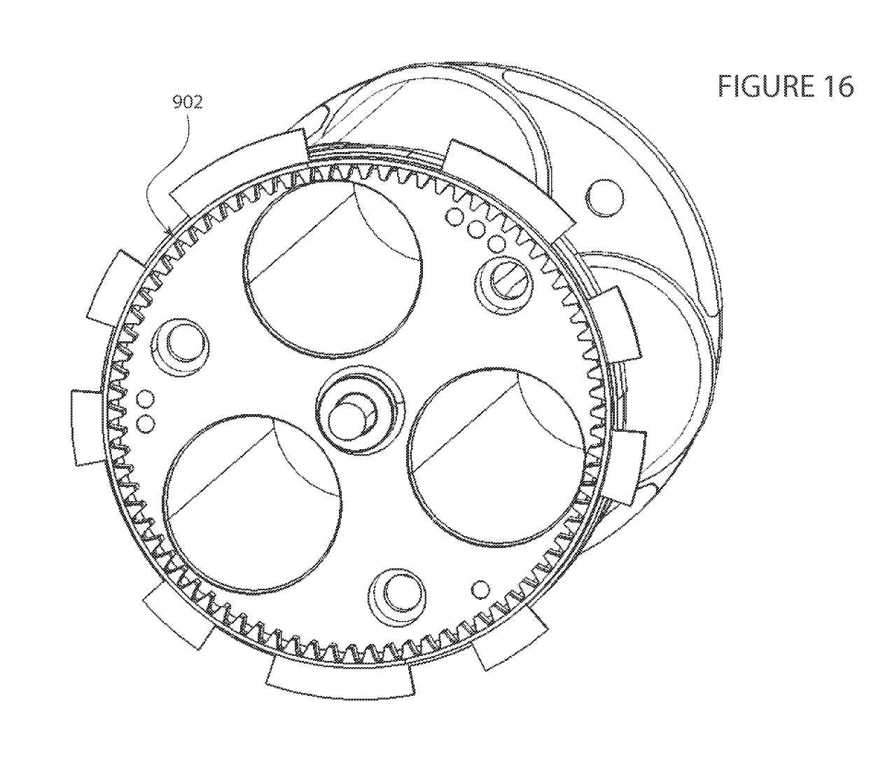

FIG. 14 illustrates a front side perspective view of an embodiment of the telescope barrel having tube support 901 that holds three tubes 1401, 1402 and 1403 that define three magnifications paths respectively. FIG. 15 illustrates a front acute perspective view of an embodiment of the telescope barrel. FIG. 16 illustrates a rear acute perspective view of an embodiment of the telescope barrel showing ring gear 902 that is utilized to rotate the magnification paths into the light path. In one or more embodiments, the barrels may be weighted or constructed of thicker or thinner material, for example thinner or thicker aluminum, to account for different weights of the optics in each discrete magnification path. For example, a lower value magnification lens or set of lenses may weigh less than a higher magnification lens or set of lenses and so the barrel that holds the lower magnification lens or set of lenses may be constructed thicker or have weights to make the barrels weigh approximately the same. In one or more embodiments the weights may be implemented at the end of the barrels to increase the moment arm of the barrel assembly and make the barrel assembly more forgiving to force perturbations derived from the hand holding the device. This makes the barrels more stable and hence the apparatus more stable during use and during barrel rotation.

One or more embodiments as shown form a light path with a focal distance of approximately 300 mm, having an eyepiece exit pupil diameter of approximately 3 mm, wherein the front housing, or second side utilizes an entrance aperture of between 8 and 12 mm. In one or more embodiments, the at least one illuminator includes at least two light emitting diodes and one filter. In other embodiments, the at least one illuminator includes at least two filters and one light emitting diode, wherein one filter may be clear or not for example. In one or more embodiments the illuminators provide two or more wavelength ranges includes a first wavelength range of 400-650 nm, and a second wavelength range of 315 and 700 nm with respect to internal transmittance of at least 1%. In other embodiments, the two or more wavelength ranges includes a first wavelength range of 425-625 nm, and a second wavelength range of 600-670 nm with respect to internal transmittance of at least 1%. In some embodiments, the controller is further configured to control luminous intensity of the at least one illuminator, for example to provide different output levels. Other embodiments may sense ambient or received light intensity and adjust the output level of the illuminators accordingly to provide fixed intensity output levels. In one or more embodiments, the at least one illuminator may output a luminosity of at least 5000 Lux, or 7500 Lux, or 9000 Lux, or any other value for example depending on the particular desired operational characteristics of the intended implementation. In one implementation PHILIPS® LUXEON® REBEL ES® LED's are utilized, with part number LXW9-PW30, having a temperature profile of 3000K, with a color rendering index of 90-95, minimum flux of at least 120 lumens.

In one or more embodiments, the first of the two or more discrete magnification level paths may utilize a magnification level of 5 power and a second of the two or more discrete magnification level paths may utilize a magnification level other than 5 power. Other embodiments may utilize three or more discrete magnification level paths wherein a first of the three or more discrete magnification level paths includes a magnification level of 5 power and a second of the three or more discrete magnification level paths includes a magnification level of 8 power and a third of the three or more discrete magnification level paths includes a magnification level of 12 power. Any other values may be utilized for magnification levels in two, three or any number of fixed powers greater than three in keeping with the spirit of the invention.

Embodiments may implement three or more discrete magnification level paths that include a first depth of focus of approximately 9 mm, approximately 5 mm, and approximately 2.5 mm at 10% contrast respectively. Embodiments may implement three or more discrete magnification level paths that include a first separation between discernable points of 40 um or less, 25 um or less, 17 um or less at 10% contrast respectively.

Embodiments of the two or more discrete magnification level paths may utilize a curved focal plane configured to approximate a typical curvature of the cervix to enable a more robust viewing of the cervix when the apparatus is held by hand. In one embodiment, the curved focal plane configured with a curvature having a radius of approximately 400 mm.

While the invention herein disclosed has been described by means of specific embodiments and applications thereof, numerous modifications and variations could be made thereto by those skilled in the art without departing from the scope of the invention set forth in the claims.

What is claimed is:

1. A portable battery powered self-illuminated multispectral multi-magnification apparatus comprising:
a housing having
a first end,
a second end and
a light path between said first end and said second end;
an eyepiece coupled with said first end and configured to enable said light path to be viewed near said first end;
a battery;
at least one illuminator coupled with said second end and said battery, wherein said at least one illuminator comprises two or more wavelength ranges and wherein said at least one illuminator is configured to project light away from said second end towards a portion of a body of a patient;
two or more discrete magnification level paths situated within said housing wherein each of said two or more discrete magnification level paths are configured to receive and pass said light that is reflected from said portion of said body of said patient to said eyepiece when a selected magnification level path is located in said light path; and,
a controller coupled with said at least one illuminator and said two or more discrete magnification level paths wherein said controller is configured to
receive user input to
control said at least one illuminator and
switch between said two or more discrete magnification level paths.

2. The portable battery powered self-illuminated multispectral multi-magnification apparatus of claim 1, wherein said light path comprises a focal distance of approximately 300 mm.

3. The portable battery powered self-illuminated multispectral multi-magnification apparatus of claim 1, wherein said eyepiece comprises an exit pupil diameter of approximately 3 mm.

4. The portable battery powered self-illuminated multispectral multi-magnification apparatus of claim 1, wherein said second side comprises an entrance aperture of between 8 and 12 mm.

5. The portable battery powered self-illuminated multispectral multi-magnification apparatus of claim 1, wherein said at least one illuminator comprises at least one light emitting diodes with an adjustable output frequency spectrum.

6. The portable battery powered self-illuminated multispectral multi-magnification apparatus of claim 1, wherein said at least one illuminator comprises at least two light emitting diodes and one filter.

7. The portable battery powered self-illuminated multispectral multi-magnification apparatus of claim 1, wherein said at least one illuminator comprises at least one filter and one light emitting diode.

8. The portable battery powered self-illuminated multispectral multi-magnification apparatus of claim 1, further comprising a filter configured to filter at least a portion of Red light from said at least one illuminator or pass at least a portion Blue and Green light from said at least one illuminator.

9. The portable battery powered self-illuminated multispectral multi-magnification apparatus of claim 1 wherein said two or more discrete magnification level paths are configured as two or more barrels that are configured to rotate within said housing to so that said selected magnification level path is located in said light path.

10. The portable battery powered self-illuminated multispectral multi-magnification apparatus of claim 1 wherein said two or more discrete magnification level paths are configured as two or more barrels that are configured to rotate within said housing to so that said selected magnification level path is located in said light path and wherein said two or more barrels are configured to weigh an approximately equal amount.

11. The portable battery powered self-illuminated multispectral multi-magnification colposcope of claim 1, wherein said controller is further configured to control luminous intensity of said at least one illuminator.

12. The portable battery powered self-illuminated multispectral multi-magnification apparatus of claim 1, wherein said at least one illuminator comprises a luminosity of at least 5000 Lux.

13. The portable battery powered self-illuminated multispectral multi-magnification apparatus of claim 1, wherein said at least one illuminator comprises a luminosity of at least 7500 Lux.

14. The portable battery powered self-illuminated multispectral multi-magnification apparatus of claim 1, wherein a first of said two or more discrete magnification level paths comprises a magnification level of 5 power and a second of said two or more discrete magnification level paths comprises a magnification level other than 5 power.

15. The portable battery powered self-illuminated multispectral multi-magnification apparatus of claim 1, wherein one of said two or more discrete magnification level paths comprises three or more discrete magnification level paths wherein a first of said three or more discrete magnification level paths comprises a magnification level of 5 power and a second of said three or more discrete magnification level paths comprises a magnification level of 8 power and a third of said three or more discrete magnification level paths comprises a magnification level of 12 power.

16. The portable battery powered self-illuminated multispectral multi-magnification apparatus of claim 1, wherein one of said two or more discrete magnification level paths comprises three or more discrete magnification level paths wherein a first of said three or more discrete magnification level paths comprises a first depth of focus of approximately 9 mm and a second of said three or more discrete magnification level paths comprises a second depth of focus of approximately 5 mm and a third of said three or more discrete magnification level paths comprises a third depth of focus of approximately 2.5 mm at 10% contrast.

17. The portable battery powered self-illuminated multispectral multi-magnification apparatus of claim 1, wherein one of said two or more discrete magnification level paths comprises three or more discrete magnification level paths wherein a first of said three or more discrete magnification level paths comprises a first separation between discernable points of 40 um or less and a second of said three or more discrete magnification level paths comprises a second separation between discernable points of 25 um or less and a third of said three or more discrete magnification level paths comprises a third separation between discernable points of 17 um or less at 10% contrast.

18. The portable battery powered self-illuminated multispectral multi-magnification apparatus of claim 1, further comprising at least one user interface element configured to accept an operator gesture and pass said gesture to said controller to control said at least one illuminator and said two or more discrete magnification level paths wherein the user interface element may be operated with a hand of the operator that supports said housing.

19. The portable battery powered self-illuminated multispectral multi-magnification apparatus of claim 1, further comprising at least one user interface element configured to accept an operator gesture and pass said operator gesture to said controller that is configured to alter said at least one illuminator and said two or more discrete magnification level paths respectively.

20. The portable battery powered self-illuminated multispectral multi-magnification apparatus of claim 1, further comprising at least two user interface elements configured to accept an operator gesture respectively and pass said gesture to said controller, wherein said controller is configured to alter said at least one illuminator between a white mode and a Green or non-Red mode of illumination and wherein a magnification of 12 power is selected from said two or more discrete magnification level paths when said green mode of illumination is selected and wherein said magnification is not altered when said white mode is selected via said gesture.

21. The portable battery powered self-illuminated multispectral multi-magnification apparatus of claim 1, further comprising at least one user interface element configured to accept an operator gesture and pass said gesture to said controller wherein said at least one user interface element comprises a first touch sensitive area configured to transmit said gesture to said controller that is configured to increase and decrease light intensity when in a white mode of illumination based on said gesture.

22. The portable battery powered self-illuminated multispectral multi-magnification apparatus of claim 1, further comprising at least one user interface element configured to accept an operator gesture and pass said gesture to said controller wherein said at least one user interface element comprises a second touch sensitive area configured to increase and decrease magnification respectively via said controller that is configured to switch between said two or more discrete magnification level paths based on said gesture.

23. The portable battery powered self-illuminated multispectral multi-magnification apparatus of claim 1, wherein at least one of said two or more discrete magnification level paths comprises a curved focal plane configured to approximate a typical curvature of a cervix.

24. The portable battery powered self-illuminated multispectral multi-magnification apparatus of claim 1, wherein at least one of said two or more discrete magnification level paths comprises a curved focal plane configured with a curvature having a radius of approximately 400 mm.

25. The portable battery powered self-illuminated multispectral multi-magnification apparatus of claim 1, wherein said housing is no more than 50 mm wide, 83 mm high and 166 mm long and wherein said portable battery powered self-illuminated multispectral multi-magnification colposcope weighs less than 500 g.

26. A portable battery powered self-illuminated multispectral multi-magnification apparatus comprising:
a housing having
a first end,
a second end and
a light path between said first end and said second end;
an eyepiece coupled with said first end and configured to enable said light path to be viewed near said first end;
a battery;
at least one illuminator coupled with said second end and said battery, wherein said at least one illuminator comprises two or more wavelength ranges and wherein said at least one illuminator is configured to project light away from said second end towards a cervix of a patient;
a filter configured to filter at least a portion of Red light from said at least one illuminator or pass at least a portion Blue and Green light from said at least one illuminator;
two or more discrete magnification level paths situated within said housing wherein each of said two or more discrete magnification level paths are configured to receive and pass said light that is reflected from said cervix of said patient to said eyepiece when a selected magnification level path is located in said light path wherein at least one of said two or more discrete magnification level paths comprises a curved focal plane configured to approximate a typical curvature of the cervix;
a controller coupled with said at least one illuminator and said two or more discrete magnification level paths wherein said controller is configured to
receive user input to
control said at least one illuminator and
switch between said two or more discrete magnification level paths; and,
at least one user interface element configured to accept an operator gesture and pass said gesture to said controller to control said at least one illuminator and said two or more discrete magnification level paths wherein the user interface element may be operated with a hand of the operator that supports said housing.

27. The portable battery powered self-illuminated multispectral multi-magnification as claimed in claim 1, wherein said portion of said body is a cervix.

28. The portable battery powered self-illuminated multispectral multi-magnification as claimed in claim 1, wherein said portion of said body is a body cavity.

29. The portable battery powered self-illuminated multispectral multi-magnification as claimed in claim 1, wherein said portion of said body is tissue.
